(19) United States
(12) Patent Application Publication
(10) Pub. No.: US 2018/0098685 A1
(43) Pub. Date: Apr. 12, 2018

(54) ENDOSCOPE APPARATUS

(71) Applicant: OLYMPUS CORPORATION, Tokyo (JP)

(72) Inventor: Kenro Osawa, Tokyo (JP)

(73) Assignee: OLYMPUS CORPORATION, Tokyo (JP)

(21) Appl. No.: 15/838,652

(22) Filed: Dec. 12, 2017

Related U.S. Application Data
(63) Continuation of application No. PCT/JP2015/069590, filed on Jul. 8, 2015.

(51) Int. Cl. A61B 1/00; A61B 1/045

(52) U.S. Cl. CPC A61B 1/00009; H04N 2005/2255; A61B 1/045; A61B 1/0005

(57) ABSTRACT

A plurality of images of an observation target are consecutively acquired with time intervals, coordinates of an observation position are identified in each image, and a plurality of corresponding points, which are pixel positions at which a image and a previously captured image correspond, are detected, wherein in this endoscope apparatus, when the coordinates of the observation position cannot be identified in the image, the coordinates of the observation position identified in the previously captured image are transformed to coordinates in a coordinate system of the image, and a direction of the transformed coordinates of the observation position with respect to the image center is calculated and is displayed together with the image.

### CROSS-REFERENCE TO RELATED APPLICATIONS

This application is a Continuation Application of International Application No. PCT/JP2015/069590 filed on Jul. 8, 2015. The content of International Application No. PCT/JP2015/069590 is incorporated herein by reference in its entirety.

### TECHNICAL FIELD

The present invention relates to endoscope apparatuses.

### BACKGROUND ART

There is a known endoscope apparatus which has a long, thin insertion portion which is inserted into a narrow space, and which captures an image of a desired area of an observation target located inside the space with an image acquisition unit provided at the distal end of the insertion portion for observation (for example, see PTL 1 and PTL 2).

### CITATION LIST

Patent Literature

{PTL 1} Japanese Unexamined Patent Application, Publication No. 2012-245161

{PTL 2} Japanese Unexamined Patent Application, Publication No. 2011-152202

### SUMMARY OF INVENTION

An aspect of the present invention is an endoscope apparatus including: an image sensor that consecutively acquires a plurality of images I (t1) to I (tn) of an observation target at times t1 to tn, in which n is an integer, with time intervals; one or more processors that process the plurality of images acquired by the image sensor; and a display that displays the images processed by the one or more processors, wherein the one or more processors are configured to conduct: a corresponding-point detecting process of detecting a plurality of corresponding points, which are pixel positions at which the image I (tn) and the image I (tn-1) correspond; an observation-position identification process of identifying coordinates of an observation position in each image; and a coordinate-transformation process of transforming the coordinates of the observation position identified in the image I (tn-1) to coordinates in a coordinate system of the image I (tn) by using the plurality of corresponding points when the observation-position identification process cannot identify the coordinates of the observation position in the image I (tn), wherein the display displays, together with the image I (tn) processed by the one or more processors, information about the coordinates of the observation position in the coordinate system of the image I (tn), which is transformed by the coordinate-transformation process.

Another aspect of the present invention is an endoscope apparatus including: an image sensor that consecutively acquires a plurality of images I (t1) to I (tn) of an observation target at times t1 to tn, in which n is an integer, with time intervals; one or more processors that process the plurality of images acquired by the image sensor; and a display that displays the images processed by the one or more processors, wherein the one or more processors are configured to conduct: a corresponding-point detecting process of detecting a plurality of corresponding points, which are pixel positions at which the image I (tn) and the image I (tn-1) correspond; an observation-position identification process of calculating a separation distance between the image I (tn) and the image I (tn-1) on the basis of the plurality of corresponding points and that identifies coordinates included in the image I (tn-1) as coordinates of an observation position when the separation distance is greater than a predetermined threshold; and a coordinate-transformation process of transforming the coordinates of the observation position identified in the image I (tn-1) to coordinates in a coordinate system of the image I (tn) by using the plurality of corresponding points, wherein the display displays, together with the image I (tn) processed by the one or more processors, information about the coordinates of the observation position in the coordinate system of the image I (tn), which is transformed by the coordinate-transformation process.

### BRIEF DESCRIPTION OF DRAWINGS

FIG. 1 is a block diagram schematically showing the configuration of an endoscope apparatus according to a first embodiment of the present invention.

FIG. 2 is an explanatory diagram showing an example image acquired by the endoscope apparatus in FIG. 1.

FIG. 3 is an explanatory diagram showing example images acquired by the endoscope apparatus in FIG. 1.

FIG. 4 is an explanatory diagram showing example images acquired by the endoscope apparatus in FIG. 1.

FIG. 5 is an explanatory diagram showing example images acquired by the endoscope apparatus in FIG. 1.

FIG. 6 is an explanatory diagram showing example images acquired by the endoscope apparatus in FIG. 1.

FIG. 7 is an explanatory diagram showing example images acquired by the endoscope apparatus in FIG. 1.

FIG. 8 is an explanatory diagram showing the direction of an observation position obtained by coordinate transformation in the endoscope apparatus in FIG. 1.

FIG. 9 is a diagram for explaining determination of the direction of an arrow indicated on a guide image when the direction of the observation position is identified and the guide image is generated by the endoscope apparatus in FIG. 1.

FIG. 10 is an explanatory diagram showing an example image displayed on a display in the endoscope apparatus in FIG. 1.

FIG. 11 is a flowchart related to an operation of the endoscope apparatus in FIG. 1.

FIG. 12 is a block diagram schematically showing the configuration of an endoscope apparatus according to a second embodiment of the present invention.

FIG. 13 is an explanatory diagram showing an example image acquired by the endoscope apparatus in FIG. 12.

FIG. 14 is an explanatory diagram showing example images acquired by the endoscope apparatus in FIG. 12.

### DESCRIPTION OF EMBODIMENTS

### First Embodiment

An endoscope apparatus according to a first embodiment of the present invention will be described below with reference to the drawings. Note that, in this embodiment, an example case where the observation target is the colon, and a scope section of the endoscope apparatus is inserted into the colon will be described.

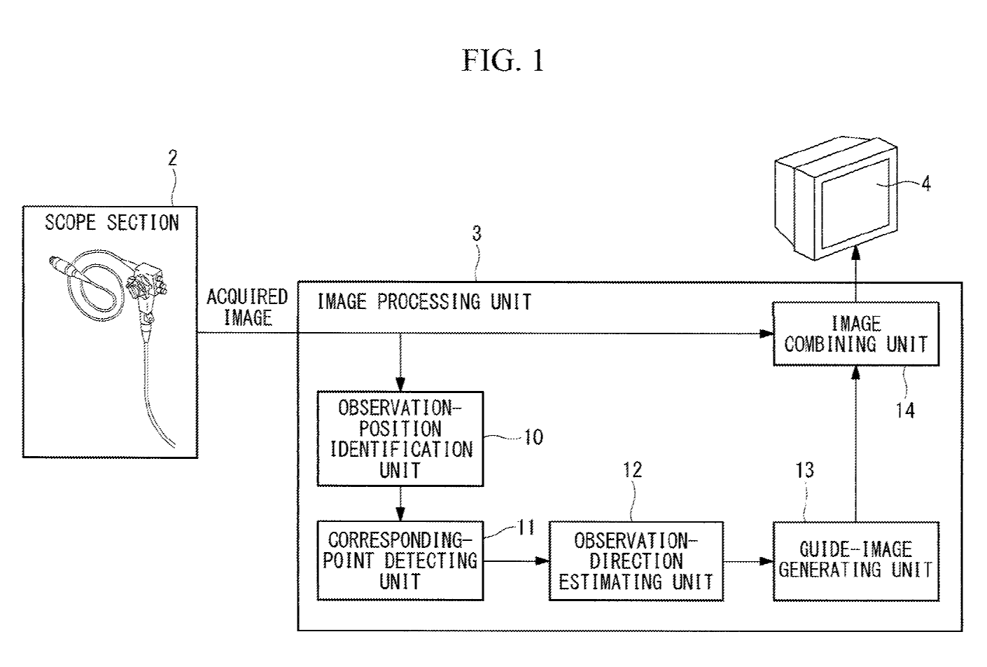

As shown in FIG. 1, an endoscope apparatus according to this embodiment includes: a flexible scope section 2 that is configured to be long, thin and that is inserted into a subject to acquire images of an observation target; an image processing unit 3 that performs predetermined processing on the images acquired by the scope section 2; and a display 4 that displays the images processed by the image processing unit 3.

The scope section 2 has, at a distal end portion thereof, a CCD serving as an image acquisition unit, and an objective lens disposed on the image-acquisition-surface side of the CCD. The scope section 2 acquires image I (t1) to image I (tn) at times t1 to tn by bending the distal end portion in a desired direction. Note that an imaging element (image sensor) can also be used as the image acquisition unit.

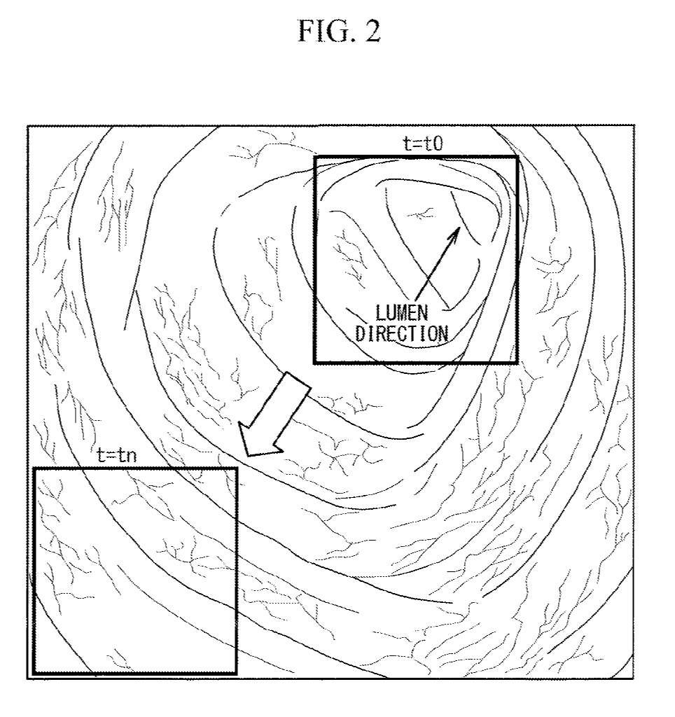

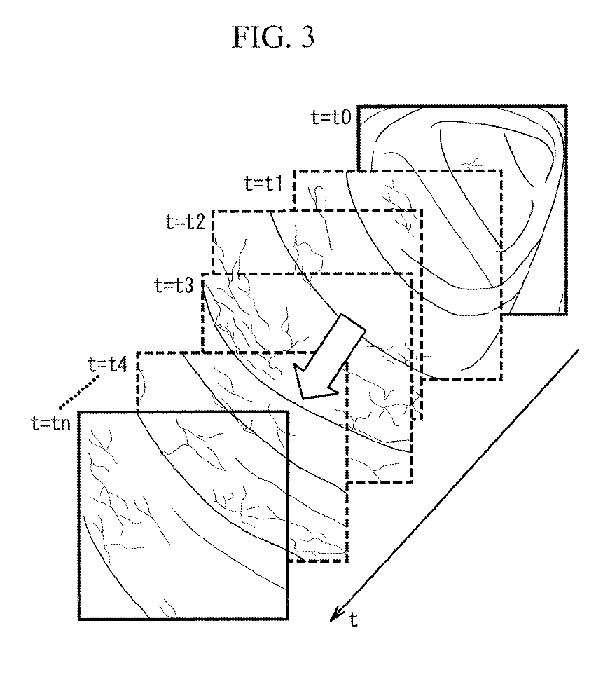

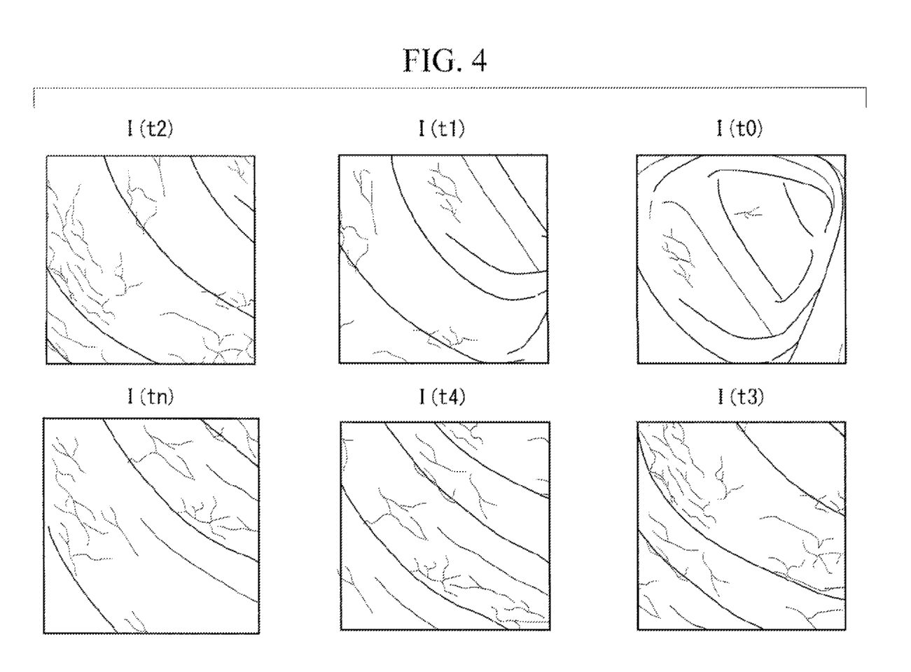

It is assumed that when, for example, the scope section 2 acquires an image of the colon, an image of an area including a deep part of the lumen of the colon is acquired at time t=t0, as shown in FIG. 2. Furthermore, it is assumed that a plurality of images are acquired at a certain frame rate as the time goes on, and an image shown in the lower left frame in FIG. 2 is acquired at time t=tn. As shown in, for example, FIGS. 3 and 4, between t=0 and t=n, images I (t1), I (t2), I (t3), I (t4) . . . I (tn) are acquired at times t=t1, t2, t3, t4 . . . tn. In the images I (t0) and I (t1), it is easy to determine the deep position of the lumen in the image. However, in the image I (tn), it is difficult to determine the deep position of the lumen in the image.

The image processing unit 3 includes an observation-position identification unit 10, a corresponding-point detecting unit 11, an observation-direction estimating unit 12 (coordinate-transformation processing unit, direction estimating unit), a guide-image generating unit 13, and an image combining unit 14. Note that the image processing unit can be configured using one or more processors which read a program and conduct processes in accordance with the program, and a memory which stores the program. Also, the image processing unit can be configured using an application specific integrated circuit (ASIC) or a field-programmable gate array (FPGA).

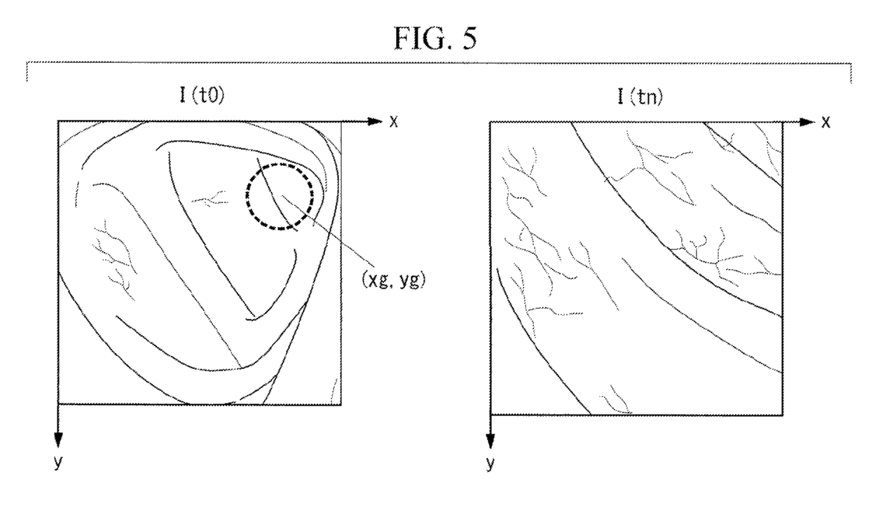

The observation-position identification unit 10 identifies the coordinates of the observation positions in the images of the observation target acquired by the scope section 2. Specifically, in the images acquired by the scope section 2 at times t1 to tn, the observation-position identification unit 10 identifies the coordinates (xg, yg) of the observation position in each image, as shown in FIG. 5.

The observation target in this embodiment is the colon, and examination or treatment is performed by inserting the scope section 2 into the colon. Accordingly, herein, the coordinates of the observation position to be identified by the observation-position identification unit 10 are at the deepest part in the direction in which the scope section 2 advances, that is, the deepest part of the lumen. The coordinates of the deepest part of the lumen can be detected by, for example, calculation based on the brightness. Specifically, the inside of the image is sectioned into predetermined local areas, and the average brightness is calculated for each local area. When the ratio of the average brightness of a local area to the average brightness of the overall image is less than or equal to a predetermined value, the center coordinates of that local area are identified as the coordinates of the deepest position of the lumen, that is, the coordinates (xg, yg) of the observation position, as shown in, for example, the left figure in FIG. 5. When coordinates are obtained in more than one local area, the center coordinates of the local area, the ratio of the average brightness of which to the average brightness of the overall image is lowest, are identified as the coordinates (xg, yg) of the observation position.

As shown in the right figure in FIG. 5, when the scope section 2 captures the intestinal wall of the colon, and an image of the wall surface is obtained, it is difficult to detect the deep part of the lumen. In this case, the local area, the ratio of the average brightness of which is less than or equal to a predetermined value cannot be obtained. Hence, the coordinates of the observation position cannot be identified, and coordinates (-1, -1) are temporarily set.

The images I (t1) to I (t) at the respective times and the identified coordinates of the observation position are associated and output to the corresponding-point detecting unit 11.

The corresponding-point detecting unit 11 detects a plurality of corresponding points, which are pixel positions at which the image I (tn) and the image I (tn-1) correspond. Specifically, upon input of the image I (tn) acquired at time t =tn and the coordinates (xg, yg) of the observation position in the image I (tn), the corresponding-point detecting unit 11 detects corresponding points between the preliminarily stored image I (tn-1) acquired at time t=tn-1 and the input image I (tn).

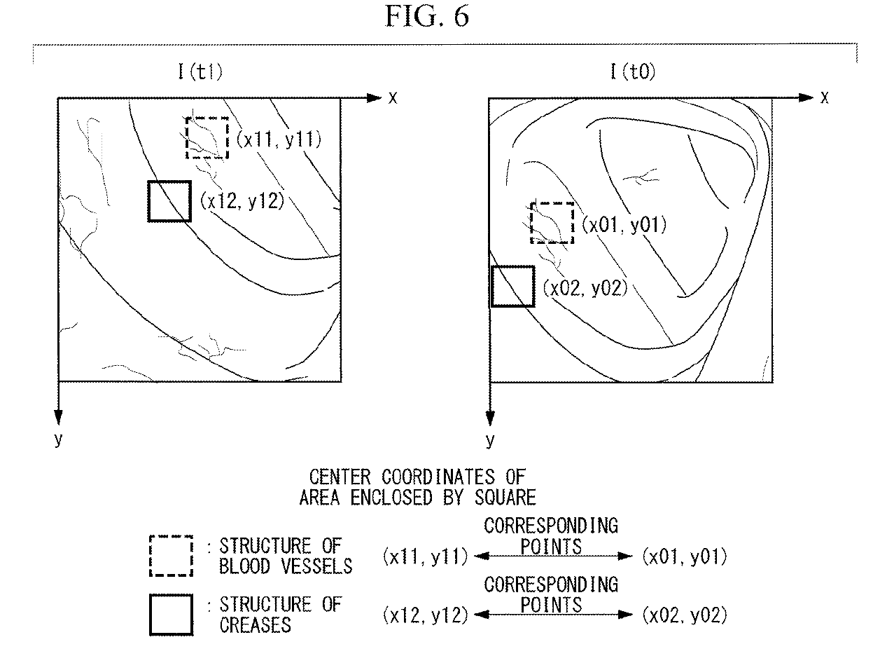

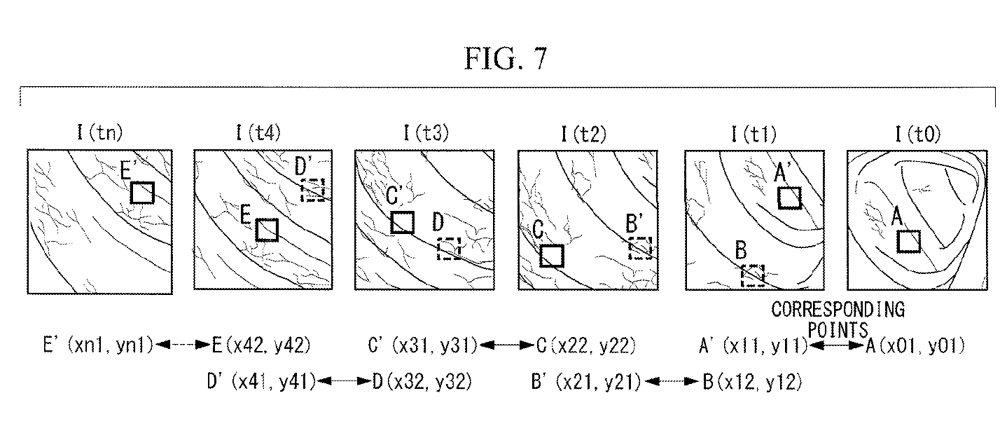

Herein, for example, as shown in FIG. 6, in each of the image I (tn) and the image I (tn-1), a pair of coordinates corresponding to the same position on the observation target are calculated as corresponding points by using image characteristics generated by the structure of blood vessels and the structure of creases included in the image as clues. Preferably, at least three corresponding points are calculated. Note that FIG. 7 shows the relationship between the corresponding points detected in a plurality of images.

Note that, when image characteristics, such as the blood vessels or the creases, cannot be identified due to image blurring or the like, it is impossible to detect the corresponding points. In that case, for example, when corresponding points cannot be set at time tn, the preliminarily stored corresponding points at time tn-1 are set as the corresponding points at time tn. This processing enables corresponding points to be set based on an assumption that a movement similar to that at time tn-1 occurs, even when the corresponding points cannot be set.

The corresponding-point detecting unit 11 stores the image I (tn) and the set corresponding points and outputs them to the observation-direction estimating unit 12.

When the observation-position identification unit 10 cannot identify the coordinates of the observation position in the image I (tn), the observation-direction estimating unit 12 transforms the coordinates of the observation position identified in the image I (tn-1) to coordinates in the coordinate system of the image I (tn) by using the plurality of corresponding points. Specifically, the coordinates (xg, yg) of the observation position in the image I (tn) and the corresponding points are input from the observation-position identification unit 10 to the observation-direction estimating unit 12, via the corresponding-point detecting unit 11.

When the coordinates (-1, -1) of the observation position in the image I (tn) are input from the observation-position identification unit 10, it is assumed that the coordinates of the observation position could not be identified, and the coordinates of the observation position identified in the preliminarily stored image I (tn-1) are transformed to coordinates (xg′, xy′) in the coordinate system of the image I (tn). Note that, when the observation position is identified, the coordinates of the observation position are stored without this transformation processing.

Here, to transform the coordinates of the observation position identified in the image I (tn-1) to coordinates in the coordinate system of the image I (tn), a coordinate transformation matrix M such as Expression (1) below is generated.

$$\begin{matrix}\{ Expression 1 \} & \\ [ \begin{matrix}x 1 \\ y 1 \\ 1\end{matrix} ] = [ \begin{matrix}m 11 & m 12 & m 13 \\ m 21 & m 22 & m 23 \\ 0 & 0 & 1\end{matrix} ] [ \begin{matrix}x 0 \\ y 0 \\ 1\end{matrix} ] & ( 1 )\end{matrix}$$

As shown by Expression (1) above, the coordinates (x0, y0) in the image before transformation are transformed to the coordinates (x1, y1). Furthermore, mij (i=1 to 2, j=1 to 3) is calculated using three or more corresponding points, by employing a least-squares method or the like.

With the thus-obtained matrix, the coordinates (xg, yg) of the observation position identified in the image I (tn-1) are transformed to the coordinates (xg′, yg′) in the coordinate system of the image I (tn), and the transformed coordinates (xg′, yg′) are stored.

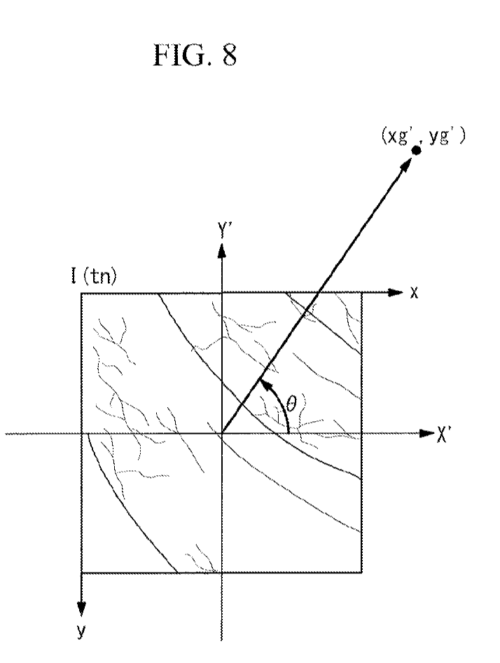

Moreover, the observation-direction estimating unit 12 calculates the direction of the transformed coordinates of the observation position with respect to the image center. More specifically, as shown in FIG. 8, the coordinates (xg′, yg′) are transformed to coordinates in the polar coordinate system, in which the center position of the image is regarded as the center coordinates, the lumen direction θ as viewed from the image center is calculated, and θ is output to the guide-image generating unit 13.

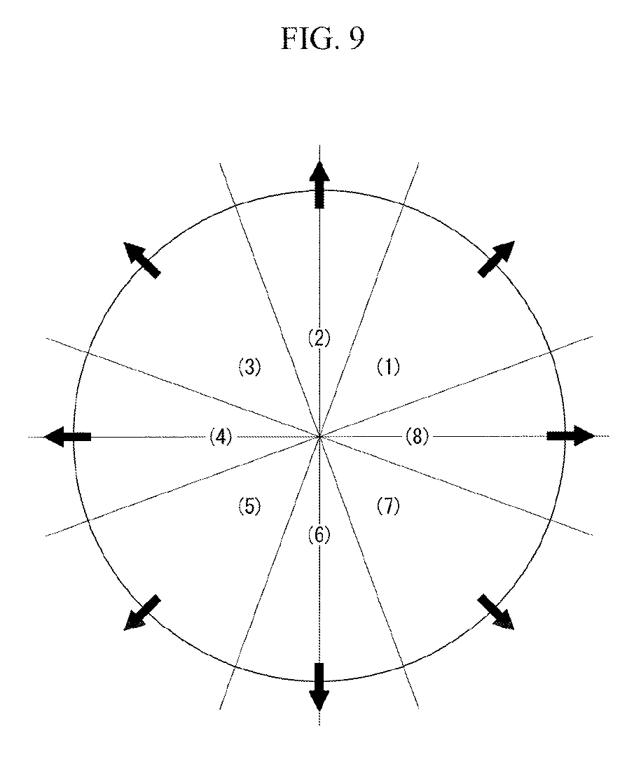

The guide-image generating unit 13 generates a guide image in which the direction indicated by θ is shown as, for example, an arrow on the image, on the basis of θ output from the observation-direction estimating unit 12. The guide-image generating unit 13 can determine the direction of the arrow to be indicated on the guide image on the basis of, for example, the area, among areas (1) to (8), to which θ belongs, in a circle sectioned into equal areas (1) to (8), as shown in FIG. 9. The guide-image generating unit 13 outputs the generated guide image to the image combining unit 14.

The image combining unit 14 combines the guide image input from the guide-image generating unit 13 and the image I (tn) input from the scope section 2 such that they overlap each other and outputs the image to the display 4.

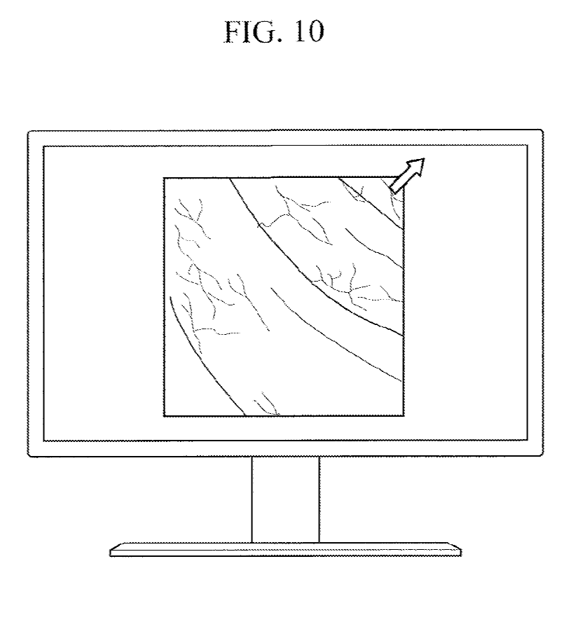

As shown in, for example, FIG. 10, an arrow indicating the direction of the lumen is indicated on the display 4, together with the image of the observation target.

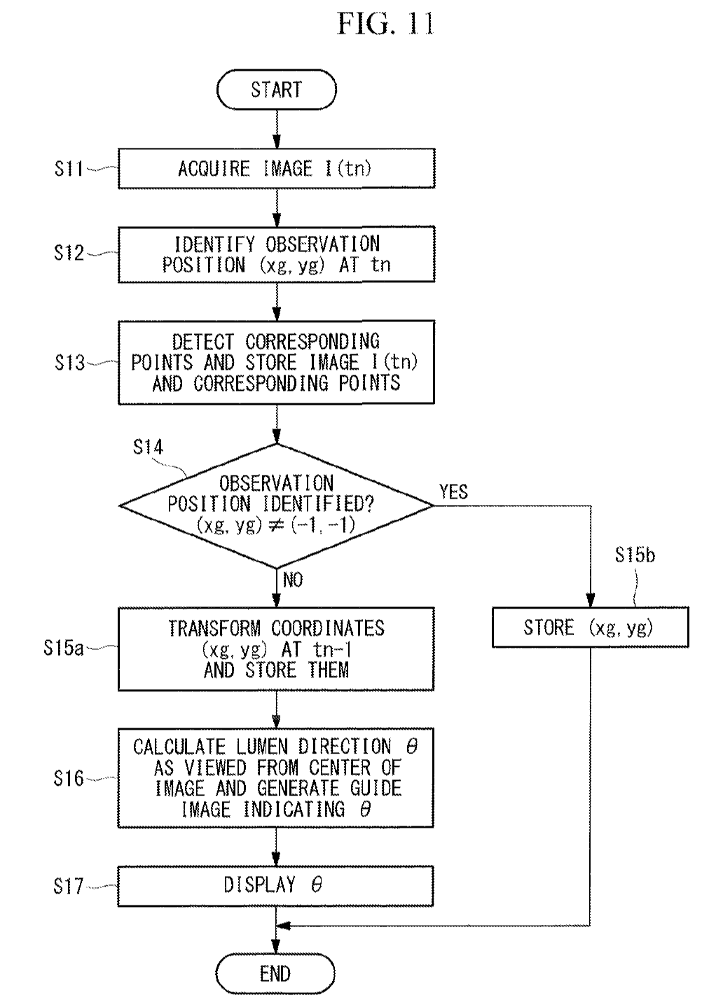

A flow of processing when the direction of the observation position is indicated in the thus-configured endoscope apparatus will be described below in accordance with the flowchart in FIG. 11.

In step S11, the scope section 2 acquires the image I (tn) at time tn, and the process proceeds to step S12.

In step S12, the coordinates (xg, yg) of the observation position are identified in the image of the observation target acquired by the scope section 2 in step S11.

As described above, the observation target in this embodiment is the colon, and the coordinates of the observation position to be identified by the observation-position identification unit 10 here are at the deepest position in the lumen. Hence, the image is sectioned into predetermined local areas, and the average brightness is calculated for each local area. When the ratio of the average brightness of a local area to the average brightness of the overall image is less than or equal to a predetermined value, the center coordinates of that local area are identified as the coordinates of the deepest position of the lumen, that is, for example, the center coordinates of the circular area indicated by a dashed line in the left figure in FIG. 5 are identified as the coordinates (xg, yg) of the observation position.

When the coordinates of the observation positions are obtained in more than one local area, the center coordinates of the local area, the ratio of the average brightness of which to the average brightness of the overall image is lowest, are identified as the coordinates (xg, yg) of the observation position. The image I (tn) and the identified coordinates of the observation position are associated and output to the corresponding-point detecting unit 11.

In step S12, when it is determined that the observation position cannot be identified, that is, as shown in the right figure in FIG. 5, when the scope section 2 captures the intestinal wall of the colon, and the obtained image is an image of the wall surface, detection of the deep part of the lumen is difficult. In that case, the local area, the ratio of the average brightness of which is less than or equal to a predetermined value cannot be obtained. Hence, the coordinates of the observation position cannot be identified, and coordinates (-1, -1) are temporarily set.

In step S13, the corresponding-point detecting unit 11 detects a plurality of corresponding points, which are pixel positions at which the image I (tn) and the image I (tn-1) correspond. Specifically, upon input of the image I (tn) acquired at time t=tn and the coordinates (xg, yg) of the observation position in the image I (tn), the corresponding-point detecting unit 11 detects the corresponding points between the preliminarily stored image I (tn-1) acquired at time t=tn-1 and the input image I (tn), and then stores the image I (tn) and the detection results.

In step S14, whether the observation position can be identified or not in step S12 is determined. When the observation position can be identified, the process proceeds to step S15b, and the observation position is stored.

When the observation position cannot be identified, the process proceeds to step S15a, and the coordinates (xg, yg) of the observation position in the preliminarily stored image I (tn-1) are transformed to the coordinates (xg′, yg′) in the coordinate system of the image I (tn).

Moreover, in step S16, the coordinates (xg′, yg′) are transformed to coordinates in the polar coordinate system, in which the center position of the image is regarded as the center coordinates, the lumen direction θ as viewed from the image center is calculated, and a guide image in which the direction indicated by θ is indicated as, for example, an arrow on the image is generated. In step S17, the image I (tn) input from the scope section 2 and the guide image are combined so as to overlap each other and are output to the display 4. On the display 4, for example, as shown in FIG. 10, the arrow indicating the direction of the lumen is indicated together with the image of the observation target.

As has been described, in this embodiment, even when the scope section 2 misses the observation target or loses the insertion direction, it is possible to quickly find the observation area or the insertion direction, and thus, to reduce the time to restart the original task and improve convenience.

Although this embodiment is configured such that a guide image is generated, in which the lumen direction θ as viewed from the image center is calculated from the coordinates (xg′, yg′) of the observation position and is indicated as an arrow on the image, and the image I (tn) and the guide image are combined so as to overlap each other and are output to the display 4, any output method may be used as long as it is possible to show the positional relationship between the image I (tn) and the coordinates (xg′, yg′) of the observation position. For example, the image I (tn) may be displayed in a small size, and the small image I (tn) and a mark indicating the position of the coordinates (xg′, yg′) of the observation position may be combined and displayed. Furthermore, in another example, it is possible to calculate the distance r from the image center from the coordinates (xg′, yg′), to generate an arrow having a length proportional to r as the guide image, and to combine the guide image with the image I (tn) to be displayed.

### Second Embodiment

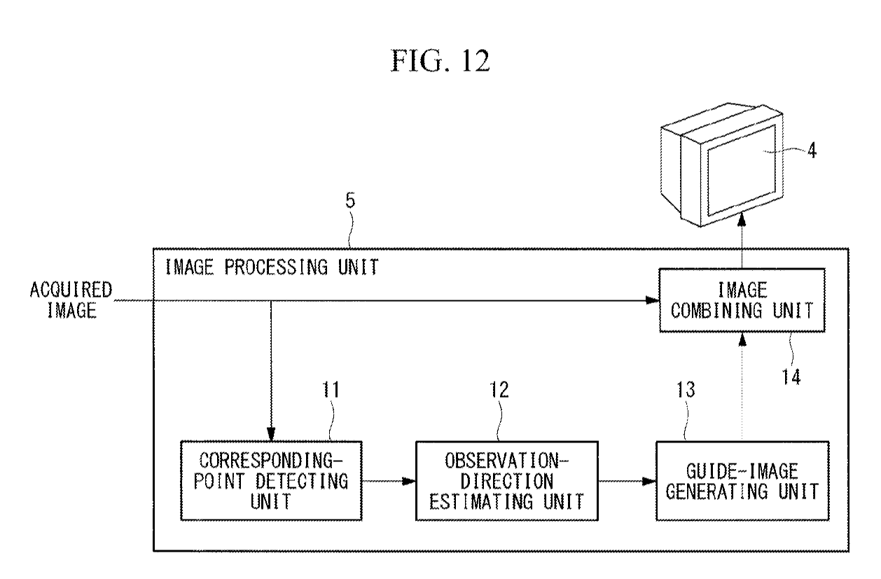

An endoscope apparatus according to a second embodiment of the present invention will be described below with reference to the drawings. In the endoscope apparatus according to this embodiment shown in FIG. 12, the components the same as those in the above-described first embodiment will be denoted by the same reference signs, and descriptions thereof will be omitted.

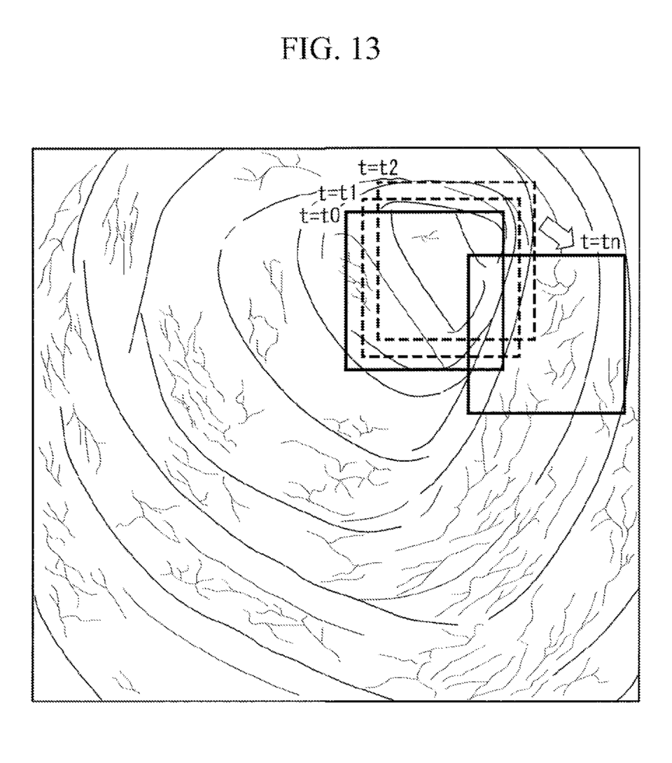

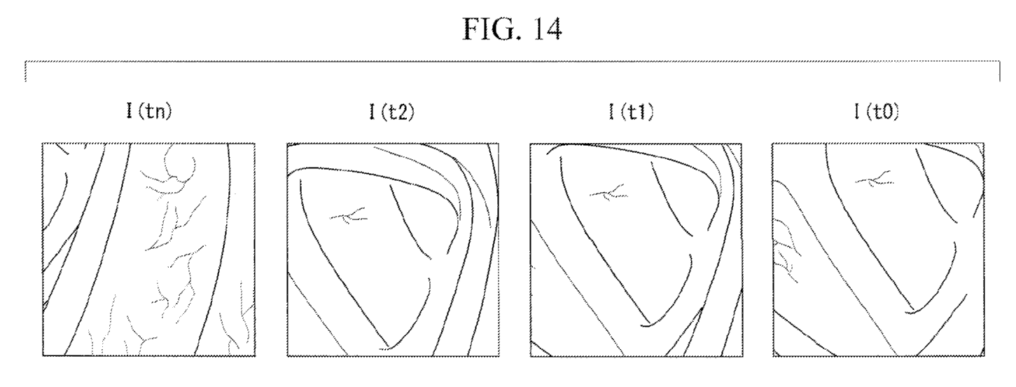

As shown in FIGS. 13 and 14, in the image processing unit 5 of the endoscope apparatus according to this embodiment, when the observation target is the colon, the scope section 2 acquires a plurality of images at a certain frame rate as the time goes on, and images I (t0), I (t1), I (t2), I (t3), I (t4) . . . I (tn) are acquired at times t=t0, t1, t2, t3, t4 . . . tn.

Although the images acquired at times t0, t1, and t2 are acquired while movement of the scope section 2 is relatively small, there is a large movement between images acquired at times t2 and tn. In other words, there are few corresponding points between the image I (t2) and the image I (tn). In this case, it is considered that an unintended abrupt change occurs, making it difficult to determine the deep position of the lumen.

To counter this problem, a guide image is generated by assuming that the center coordinates of the image I (tn-1) which is acquired immediately before the large movement occurs are the coordinates (xg, yg) of the observation position.

Specifically, the image processing apparatus 5 includes the corresponding-point detecting unit 11, the observation-direction estimating unit 12 (coordinate-transformation processing unit, direction estimating unit), the guide-image generating unit 13, and the image combining unit 14.

The corresponding-point detecting unit 11 detects a plurality of corresponding points, which are pixel positions at which the image I (tn) and the image I (tn-1) correspond. Specifically, upon input of the image I (tn) acquired at time t =tn, the corresponding-point detecting unit 11 detects corresponding points between the preliminarily stored image I (tn-1) acquired at time t=tn-1 and the input image I (tn).

Furthermore, the separation distance between the image I (tn) and the image I (tn-1) is calculated on the basis of the plurality of corresponding points, and, when the separation distance is greater than a predetermined threshold, the center coordinates of the image I (tn-1) are identified as the coordinates (xg, yg) of the observation position. The identified coordinates (xg, yg) of the observation position are output to the observation-direction estimating unit 12, together with the detected corresponding points. The corresponding-point detecting unit 11 stores the image I (tn) and the corresponding points in the corresponding-point detecting unit 11.

The observation-direction estimating unit 12 transforms, using the plurality of corresponding points, the coordinates of the observation position identified in the image I (tn-1) to coordinates in the coordinate system of the image I (tn), and calculates the direction of the transformed coordinates of the observation position with respect to the image center. Because the processing performed by the observation-direction estimating unit 12 is the same as that in the first embodiment, a detailed description thereof will be omitted here.

With the thus-configured endoscope apparatus, when it is determined, from the acquired image, that an abrupt change has occurred, it can be determined that the observation position is missing due to an unintended abrupt change. Because it is possible to estimate the direction of the observation position from the image before it is determined that the observation position is missing, it is possible to quickly find the observation area or the insertion direction, and thus, to reduce the time to restart the original task and improve convenience.

Although this embodiment is configured such that a guide image is generated by assuming the center coordinates of the image I (tn-1) immediately before a large movement to be the coordinates (xg, yg) of the observation position, for the coordinates (xg, yg) that are assumed to be the observation position, any position whose coordinates are included in the image I (tn-1) may be used as the coordinates (xg, yg). For example, in positions in the image I (tn-1), a position closest to the image I (tn) may be used as the coordinates (xg, yg).

### (Modification)

In the above-described embodiments, although the description has been given based on an assumption that the observation target is the colon, the observation target is not limited to the colon and may be, for example, an affected part of an organ. In that case, the processing can be continued by, for example, detecting an area of interest including an affected part in which any property is different from that of the peripheral parts, from the image acquired by the scope section 2 and identifying the center pixel of this area of interest as the coordinates of the observation position.

Furthermore, the observation targets are not limited to those in the medical field, and the present invention may be applied to observation targets in the industrial field. For example, when an endoscope is used to inspect a crack or the like in a pipe, by setting the crack in the pipe as the observation target, the same processing as above may be used.

As an example method for detecting an area of interest when an affected part is regarded as the area of interest, a detecting method in which the area of interest is classified according to the area and the magnitude of the color (for example, red) intensity difference from the peripheral part may be employed. Then, the same processing as that in the above-described embodiments is performed, a guide image indicating the direction of the area of interest including the affected part is generated when a guide image is generated, and an image in which the guide image is superposed on the observation image is displayed on the display 4. By doing so, it is possible to quickly show an observer an observation area and an insertion direction. Thus, it is possible to reduce the time to restart the original task, and thus, to improve convenience.

The inventor has arrived at the following aspects of the present invention.

An aspect of the present invention is an endoscope apparatus including: an image acquisition unit that consecutively acquires a plurality of images I (t1) to I (tn) of an observation target at times t1 to tn (n is an integer) with time intervals; an image processing unit that processes the plurality of images acquired by the image acquisition unit; and a display that displays the images processed by the image processing unit, wherein the image processing unit includes: a corresponding-point detecting unit that detects a plurality of corresponding points, which are pixel positions at which the image I (tn) and the image I (tn-1) correspond; an observation-position identification unit that identifies coordinates of an observation position in each image; and a coordinate-transformation processing unit that transforms the coordinates of the observation position identified in the image I (tn-1) to coordinates in a coordinate system of the image I (tn) by using the plurality of corresponding points when the observation-position identification unit cannot identify the coordinates of the observation position in the image I (tn), wherein the display displays, together with the image I (tn) processed by the image processing unit, information about the coordinates of the observation position in the coordinate system of the image I (tn), which is transformed by the coordinate-transformation processing unit.

According to this aspect, the corresponding-point detecting unit detects a plurality of corresponding points, which are pixel positions at which the image I (tn) and the image I (tn-1) correspond, in the plurality of images acquired by the image acquisition unit, and the observation-position identification unit identifies the coordinates of the observation position in each image. This processing is sequentially repeated, and when the coordinates of the observation position cannot be identified in the image I (tn), the coordinate-transformation processing unit transforms the coordinates of the observation position identified in the image I (tn-1) to coordinates in the coordinate system of the image I (tn) by using the plurality of corresponding points between the image I (tn) and the image I (tn-1).

When the coordinates of the observation position cannot be identified in the image I (tn), it is considered that the observation position is not included in the image I (tn), that is, the observation position is missing. In that case, by transforming the coordinates of the observation position identified in the image I (tn-1) to coordinates in the coordinate system of the image I (tn) by using the plurality of corresponding points between the image I (tn) and the image I (tn-1), it is possible to estimate the positional relationship between the image I (tn) and the image I (tn-1).

As a result, it is possible to calculate and estimate the direction in which the coordinates of the observation position are located, as viewed from the image I (tn). By indicating, together with the image I (tn) in which the coordinates of the observation position cannot be identified, the estimated direction as the information about the coordinates of the observation position in the coordinate system of the image I (tn), even when the observation position is not included in the image I (tn), it is possible to show a user the direction in which the observation position is located, as viewed from the image I (tn). Thus, the user can quickly find the observation area or the insertion direction and thus can reduce the time to restart the original task, even when the user misses the observation target or loses the insertion direction.

Note that, by providing the direction estimating unit that calculates the direction of the coordinates of the observation position transformed by the coordinate-transformation processing unit with respect to the image center, it is possible to calculate, with the direction estimating unit, the direction of the transformed coordinates of the observation position with respect to the image center and to calculate and estimate the direction in which the coordinates of the observation position are located, as viewed from the image I (tn).

Another aspect of the present invention is an endoscope apparatus including: an image acquisition unit that consecutively acquires a plurality of images I (t1) to I (tn) of an observation target at times t1 to tn (n is an integer) with time intervals; an image processing unit that processes the plurality of images acquired by the image acquisition unit; and a display that displays the images processed by the image processing unit, wherein the image processing unit includes: a corresponding-point detecting unit that detects a plurality of corresponding points, which are pixel positions at which the image I (tn) and the image I (tn-1) correspond; an observation-position identification unit that calculates a separation distance between the image I (tn) and the image I (tn-1) on the basis of the plurality of corresponding points and that identifies coordinates included in the image I (tn-1) as coordinates of an observation position when the separation distance is greater than a predetermined threshold; and a coordinate-transformation processing unit that transforms the coordinates of the observation position identified in the image I (tn-1) to coordinates in a coordinate system of the image I (tn) by using the plurality of corresponding points, wherein the display displays, together with the image I (tn) processed by the image processing unit, information about the coordinates of the observation position in the coordinate system of the image I (tn), which is transformed by the coordinate-transformation processing unit.

According to this aspect, in the plurality of images acquired by the image acquisition unit, the corresponding-point detecting unit detects a plurality of corresponding points, which are pixel positions at which the image I (tn) and the image I (tn-1) correspond, and the separation distance between the image I (tn) and the image I (tn-1) is calculated on the basis of the plurality of corresponding points. This processing is sequentially repeated, and when the separation distance is greater than a predetermined threshold, the observation-position identification unit identifies coordinates (e.g., the center coordinates) included in the image I (tn-1) as the coordinates of the observation position. When the separation distance between the image I (tn) and the image I (tn-1) is greater than the predetermined threshold, it is considered that a large movement has occurred between times tn and tn-1 and that the image acquisition unit has missed the observation position. In that case, the observation-position identification unit identifies coordinates included in the image I (tn-1) as the coordinates of the observation position, and the coordinate-transformation processing unit transforms the coordinates of the observation position to coordinates in the coordinate system of the image I (tn) by using the plurality of corresponding points between the image I (tn) and the image I (tn-1).

As a result, it is possible to estimate the positional relationship between the image I (tn) and the image I (tn-1) and to calculate and estimate the direction in which the coordinates of the observation position are located, as viewed from the image I (tn).

Moreover, by indicating the estimated direction together with the image I (tn) in which the coordinates of the observation position cannot be identified, even when the observation position is not included in the image I (tn), it is possible to show a user the direction in which the observation position is located, as viewed from the image I (tn). Thus, even when the user misses the observation target or loses the insertion direction, the user can quickly find the observation area or the insertion direction and thus can reduce the time to restart the original task.

In the above aspect, the observation-position identification unit may identify, as the coordinates of the observation position, coordinates showing a deepest position in a lumen in the observation target.

With this configuration, for example, when the observation target is the colon, and examination or treatment is performed while the endoscope is inserted into the lumen of the colon, even if the advancing direction is lost, it is possible to indicate the advancing direction. Thus, the user can quickly find the observation area or the insertion direction and restart the original task.

In the above aspect, the observation-position identification unit may identify, as the coordinates of the observation position, coordinates showing a position of an affected part in the observation target.

With this configuration, for example, even when the affected part is missing while the affected part is treated, it is possible to indicate the direction of the affected part, and the user can quickly find the area to be treated and restart the original task.

The aforementioned aspects provide an advantage in that, even when the observation target is missing or the insertion direction is lost, it is possible to quickly find the observation area or the insertion direction and to reduce the time to restart the original task, thus improving convenience.

### REFERENCE SIGNS LIST

2 scope section (image acquisition unit)
3 image processing unit
4 display
10 observation-position identification unit
11 corresponding-point detecting unit
12 observation-direction estimating unit (coordinate-transformation processing unit, direction estimating unit)
13 guide-image generating unit
14 image combining unit

1. An endoscope apparatus comprising:
an image sensor that consecutively acquires a plurality of images I (t1) to I (tn) of an observation target at times t1 to tn, in which n is an integer, with time intervals;
one or more processors that process the plurality of images acquired by the image sensor; and
a display that displays the images processed by the one or more processors,
wherein the one or more processors are configured to conduct:
a corresponding-point detecting process of detecting a plurality of corresponding points, which are pixel positions at which the image I (tn) and the image I (tn-1) correspond;
an observation-position identification process of identifying coordinates of an observation position in each image; and
a coordinate-transformation process of transforming the coordinates of the observation position identified in the image I (tn-1) to coordinates in a coordinate system of the image I (tn) by using the plurality of corresponding points when the observation-position identification process cannot identify the coordinates of the observation position in the image I (tn),
wherein the display displays, together with the image I (tn) processed by the one or more processors, information about the coordinates of the observation position in the coordinate system of the image I (tn), which is transformed by the coordinate-transformation process.

2. An endoscope apparatus comprising:
an image sensor that consecutively acquires a plurality of images I (t1) to I (tn) of an observation target at times t1 to tn, in which n is an integer, with time intervals;
one or more processors that process the plurality of images acquired by the image sensor; and
a display that displays the images processed by the one or more processors,
wherein the one or more processors are configured to conduct:
a corresponding-point detecting process of detecting a plurality of corresponding points, which are pixel positions at which the image I (tn) and the image I (tn-1) correspond;
an observation-position identification process of calculating a separation distance between the image I (tn) and the image I (tn-1) on the basis of the plurality of corresponding points and that identifies coordinates included in the image I (tn-1) as coordinates of an observation position when the separation distance is greater than a predetermined threshold; and
a coordinate-transformation process of transforming the coordinates of the observation position identified in the image I (tn-1) to coordinates in a coordinate system of the image I (tn) by using the plurality of corresponding points,
wherein the display displays, together with the image I (tn) processed by the one or more processors, information about the coordinates of the observation position in the coordinate system of the image I (tn), which is transformed by the coordinate-transformation process.

3. The endoscope apparatus according to claim 1, wherein the observation-position identification process identifies, as the coordinates of the observation position, coordinates showing a deepest position in a lumen in the observation target.

4. The endoscope apparatus according to claim 1, wherein the observation-position identification process identifies, as the coordinates of the observation position, coordinates showing a position of an affected part in the observation target.
